(19) United States
(12) Patent Application Publication
(10) Pub. No.: US 2009/0050141 A1
(43) Pub. Date: Feb. 26, 2009

(54) Pre-filled, single-use, disposable small volume medication nebulizer

(76) Inventors: Russell Wayne King, Sierra Madre, CA (US); Kenneth Michael McPeck, El Monte, CA (US)

Correspondence Address: JAMES E. BRUNTON, ESQ., P. O. BOX 29000, GLENDALE, CA, 91209, US

(21) Appl. No.: 11/894,860

(22) Filed: Aug. 21, 2007

(51) Int. Cl. A61M 11/00

(52) U.S. Cl. 128/200.18

(57) ABSTRACT

A miniaturized pre-filled, single-use, disposable, small volume medication nebulizer unit for medicinal use that delivers a mist of properly sized aerosol particles of medicament to the patient with a very high level of efficiency. The nebulizer can be effectively used in conjunction with conventional tee and mouthpiece patient interface devices as well as with more sophisticated interface devices such as dosimetric/reservoir systems, or mechanical ventilator systems.

### BACKGROUND OF THE INVENTION

1. Field of the Invention

The present invention relates generally to inhalation devices. More particularly, the invention concerns a miniaturized pre-filled, single-use, disposable, small volume nebulizer for medicinal use that delivers a mist of properly sized aerosol particles of medicament to the patient with a very high-level of efficiency.

2. Discussion of the Prior Art

In medicine, a nebulizer is defined as a device that is used to administer medication to the patient's airways in the form of a liquid mist, more properly known as an aerosol. In general the prior art devices used for producing medical aerosols fall into two categories; the small volume nebulizer (SVN), and the metered dose inhaler (MDI). The small volume nebulizer (SVN) has traditionally been the apparatus of choice for delivery of therapeutic aerosols. The delivery apparatus typically consists of a multi-use disposable or reusable nebulizer, a mouthpiece or facemask, and a pressurized gas source usually oxygen or air. The metered dose inhaler (MDI), on the other hand, typically contains the active drug, dissolved in chlorofluorcarbon (CFC) or chlorofluroalkane (CFA) propellants and excipients plus a metering valve. The drug-containing canister of the device is generally fitted to a mouthpiece actuator and spacer or valved holding chamber, and activation of the device by compressing it results in the release of a metered dose of medication.

Various types of prior art inhalers have also been offered for sale and are in wide use. Inhalers have the advantage of portability but have been criticized on the basis that patients often lack the coordination and psychomotor skills to use them properly without professional supervision. This dichotomy of available device types (nebulizers vs. inhalers) has lead to a great deal of controversy regarding which method is superior, although many experts have concluded that nebulizers and inhalers are essentially equivalent in terms of therapeutic outcomes. Accordingly, in many respects, the choice of device revolves around non-outcome-related factors such as cost, convenience, ease-of-use, safety, patient preference, patient compliance and adherence, as well as the availability of medications in one or both delivery forms. Despite alternative methodologies, it is clear that inhaled medication delivery by nebulizer is a permanent part of the treatment options for respiratory disease patients and is becoming a useful tool for systemic drug delivery as well.

This being the case, there is an abundance of plastic disposable medication nebulizers on the market, but the vast majority of these devices are essentially clones, differing from one another mainly in appearance. Functionally, they are essentially identical. The overriding similarity between all these devices is they are all supplied empty and the medication they are to nebulize must be transferred into them prior to commencement of the treatment by either the professional respiratory therapist in the hospital setting or the patient or patient's caregiver in the home setting.

Recently, various investigators and companies have sought to improve liquid nebulization. However, due to the physics of jet nebulization, the possibility of performance improvements in the jet nebulizer itself are very limited. Many of the improvements have involved electronic controlled or driven nebulizers that have improved efficiency but are also so expensive as to be out-of-reach for the typical routine nebulization purposes.

As will be discussed more fully hereinafter, there are various well recognized technical limitations to nebulizer use. These include the following:

1. Excessive patient dosing time.
2. Dose of drug delivered to the patient is undesirably affected by patient breathing parameters that may result in unacceptable variations in drug delivery dose.
3. Cleaning of the nebulizer after each use is time-consuming and frequently neglected thereby providing a possible avenue for nosocomial infection (bacteria/viral spread within a healthcare organization).
4. In a hospital environment excessive amounts of technologist time is required for each patient treatment.
5. Release of the drug to atmosphere is not only wasteful but can be detrimental to healthcare workers who breathe “second-hand” aerosol drugs.
6. Because of lengthy treatment times, patients may become fatigued and compliance is compromised.
7. Hospital use is often determined by price only, not performance.

In light of the aforementioned drawbacks, Dr. J. H. Dennis, a highly recognized aerosol researcher, has stated as follows in the Practical Handbook of Nebulizer Therapy. London, Martin Dunitz; 2004: 42-43:
“It is clear that neither pressurized metered dose MDI's, nor DPI's meet all the necessary requirements despite the enormous amounts of pharmaceutical funding which has been devoted to improvement of these devices over the past three decades.”
   It is this problem that the present invention seeks to address by providing the novel miniaturized pre-filled, single-use, disposable, small volume medication nebulizer unit of the invention.

### SUMMARY OF THE INVENTION

It is an object of the present invention to provide a novel miniaturized pre-filled, single-use, disposable, small volume medication nebulizer unit for medicinal use that delivers a mist of properly sized aerosol particles of medicament to the patient with a very high-level of efficiency.

Another object of the invention is to provide a nebulizer of the aforementioned character that comprises a unique means for packaging an inhalation drug in a preferred unit-dose, single-use disposable container that confers the benefits of unit-dose packaging while it simultaneously performs the function of highly effective drug aerosolization.

Another object of the invention is to provide a nebulizer of the character described that is small in physical size for convenience of packaging, storage, dispensing and operation.

Another object of the invention is to provide a miniaturized, pre-filled, single-use, disposable, nebulizer that can be produced in large quantities at minimal cost by conventional thermoplastic injection molding means.

Another object of the invention is to provide a miniaturized nebulizer as described in the preceding paragraphs that can be effectively used in conjunction with conventional tee and mouthpiece patient interface devices as well as with more sophisticated patient interface devices such as dosimetric/reservoir systems, or mechanical ventilator systems.

Another object of the invention is to provide a miniaturized, pre-filled, single-use, disposable, nebulizer that effectively mitigates against the possibility of self-contamination or cross-contamination due to improper cleaning of the device.

Another object of the invention is to provide a miniaturized nebulizer of the class described that effectively minimizes practitioner set-up and preparation time thereby conferring significant labor savings benefits upon healthcare organizations that employ such practitioners for the purpose of administering medicated aerosol therapy.

Another object of the invention is to provide a miniaturized, pre-filled, single-use, disposable nebulizer that effectively reduces or eliminates practitioner clean-up time following administration of the contained medication thereby conferring significant labor savings benefits upon healthcare organizations that employ such practitioners for the purpose of administering medicated aerosol therapy.

Another object of the invention is to provide a nebulizer of the type described in the preceding paragraphs that incorporates a miniature nebulizer attached to a dosimetric reservoir configuration that delivers superior patient dose consistency and repeatability even over a wide range of patient breathing parameters. This feature uniquely provides the ability to accurately predict the actual dose delivered to the patient. Such calculations may be made to within ±20% of that predicted when using the dose quantification equation discussed hereinafter.

Another object of the invention is to provide a miniaturized, pre-filled, single-use, disposable, nebulizer unit that, when combined with an appropriate dosimetric reservoir system, provides a substantial reduction of drug release to the ambient atmosphere thereby protecting caregivers and other personnel by reducing or minimizing exposure to “second-hand” aerosol drugs.

### BRIEF DESCRIPTION OF THE DRAWINGS

FIG. 1 is a generally perspective view of one form of the single dose disposable nebulizer unit of the invention with both closures in place as if filled with medication.

FIG. 2 is an enlarged, cross-sectional view taken along lines 2-2 of FIG. 1.

FIG. 3 is an exploded, generally perspective, cross-sectional view of the nebulizer unit illustrated in FIG. 2 of the drawings.

FIG. 4 is a generally perspective view illustrating the nebulizer unit of the invention interconnected with a patient delivery device.

FIG. 5 is a greatly enlarged, cross-sectional view taken along lines 5-5 of FIG. 4.

FIG. 6 is a view taken along lines 6-6 of FIG. 5.

FIG. 7 is a cross-sectional view taken along lines 7-7 of FIG. 5.

FIG. 8 is an enlarged, cross-sectional view taken along lines 8-8 of FIG. 5.

FIG. 9 is an enlarged cross-sectional view taken along lines 9-9 of FIG. 5.

FIG. 10 is a cross-sectional view taken along lines 10-10 of FIG. 5.

FIG. 11 is a fragmentary cross-sectional view of the upper portion of the nebulizer unit better illustrating the construction of the nebulizer assembly.

FIG. 12 is a generally perspective view, partly in cross section, showing one form of the nebulizer unit of the invention interconnected with a conventional mouthpiece, tee and aerosol flex tube reservoir.

FIG. 13 is an enlarged, cross-sectional view taken along lines 13-13 of FIG. 12.

FIG. 14 is a cross-sectional view taken along lines 14-14 of FIG. 13.

FIG. 15 is a cross-sectional view taken along lines 15-15 of FIG. 14.

### DESCRIPTION OF THE INVENTION

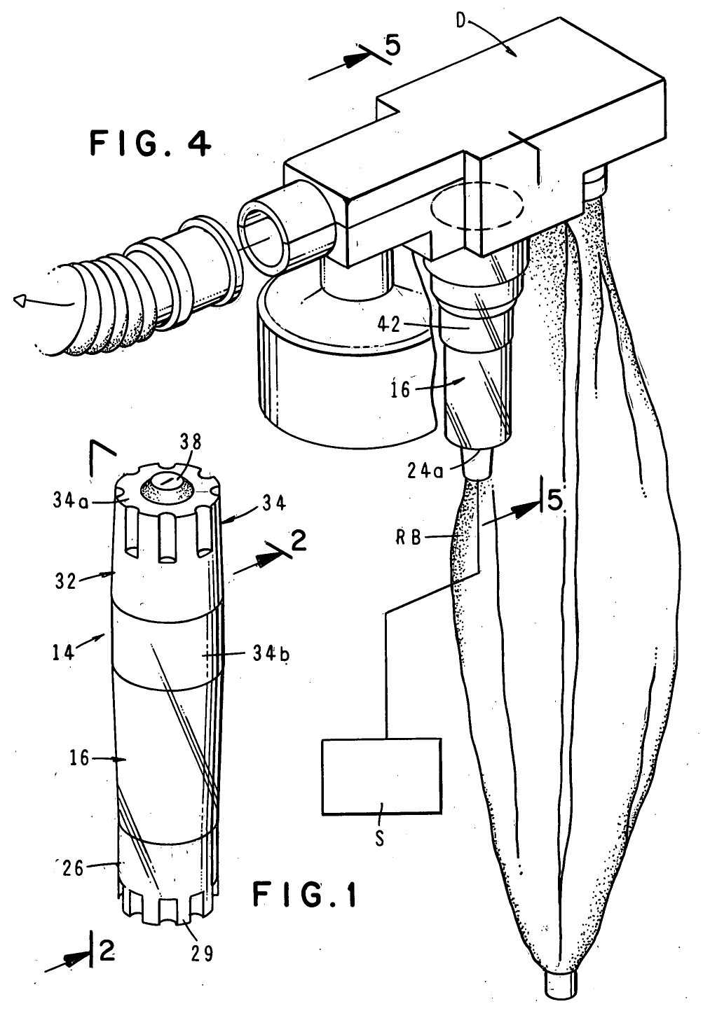

Referring to the drawings and particularly to FIGS. 1 through 3, one form of the miniaturized jet nebulizer unit of the invention for delivering a multiplicity of particles of aerosolized medication of a selected size to a patient is there illustrated and generally designated by the 14. As will be discussed more fully hereinafter, a novel feature of the nebulizer unit of the invention resides in the fact that it can be supplied pre-filled with the required inhalable liquid medication, used for a single treatment, and then discarded.

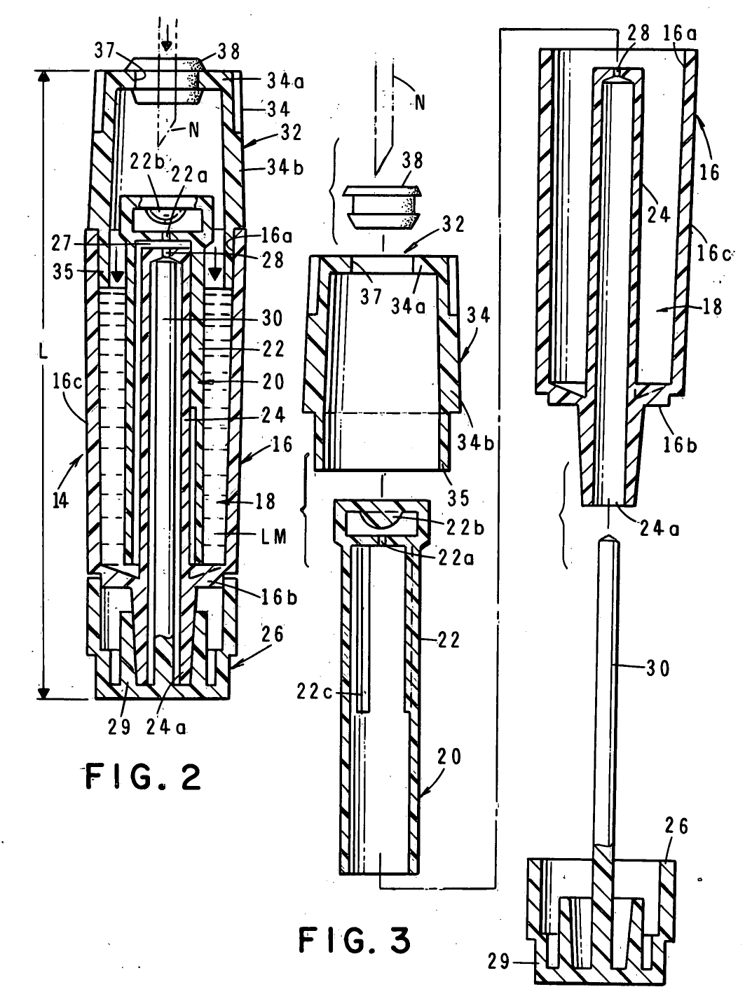

As previously mentioned, nebulizer 14 is quite small and preferably, but not limitedly, has an overall length “L” of between about 2.0 and about 3.0 inches (FIG. 2). Uniquely, nebulizer 14 serves as both the single dose package for the medication to be delivered to the patient and, in a manner presently to be described, as a means for converting the liquid medication to a respirable aerosol. In practice, the nebulizer unit 14 can be produced from a commercial polymer in very high quantities by multi-cavity thermoplastic injection molding techniques of a character well understood by those skilled in the art. The number of components that make up the nebulizer unit is intentionally minimized to facilitate molding and to enable automatic robotic assembly and testing. Inasmuch as the nebulizer unit is discarded after each treatment, its use negates the need for extensive preparation and filling prior to the treatment by healthcare professionals or home care patients. Further, because it is intended to be discarded after use, there is no need for nebulizer cleaning thereby eliminating this time-consuming step and removing any doubt about the quality and effectiveness of the cleaning process. These novel qualities of the nebulizer unit of the invention serve to significantly reduce the immense amount of professional labor time by hospital respiratory care departments and at the same time substantially reduce the possibility of iatrogenic cross-contamination via improperly cleaned nebulizers. For the pharmaceutical company, the unique design of the nebulizer unit of the invention provides a higher margin of medication safety because the need for having either a healthcare practitioner or a home care patient transfer a drug from its packaged container into the nebulizer unit is eliminated. The deliberate integral capacity limitation of the nebulizer unit for any given drug also mitigates against unauthorized admixture of the self-contained drug with other agents.

Referring particularly to FIGS. 1 through 3 of the drawings, the nebulizer unit 14 of the invention here comprises a central body 16 having first open end 16a, a second end 16b and a tapered sidewall 16c. Tapered sidewall 16c defines a fluid reservoir 18 for containing a single dose of between about 2 and about 4 milliliters of aerosolizable liquid medicament “LM”. As indicated in FIG. 11, central body 16 has a diameter “DIA” of between about 0.5 and about 0.8 inches.

Disposed within reservoir 18 for converting the aerosolizable liquid medicament into an aerosolized medication is a nebulizer assembly 20 that includes a moldable plastic nebulizer body 22 having a nebulizer orifice 22a and a deflector element 22b (FIGS. 2 and 3). Mounted within central body 16 is an elongated fluid flow tube 24 that forms a part of the nebulizer assembly of the present invention and includes a gas inlet port 24a and a gas outlet port 28 that is in communication with nebulizer orifice 22a.

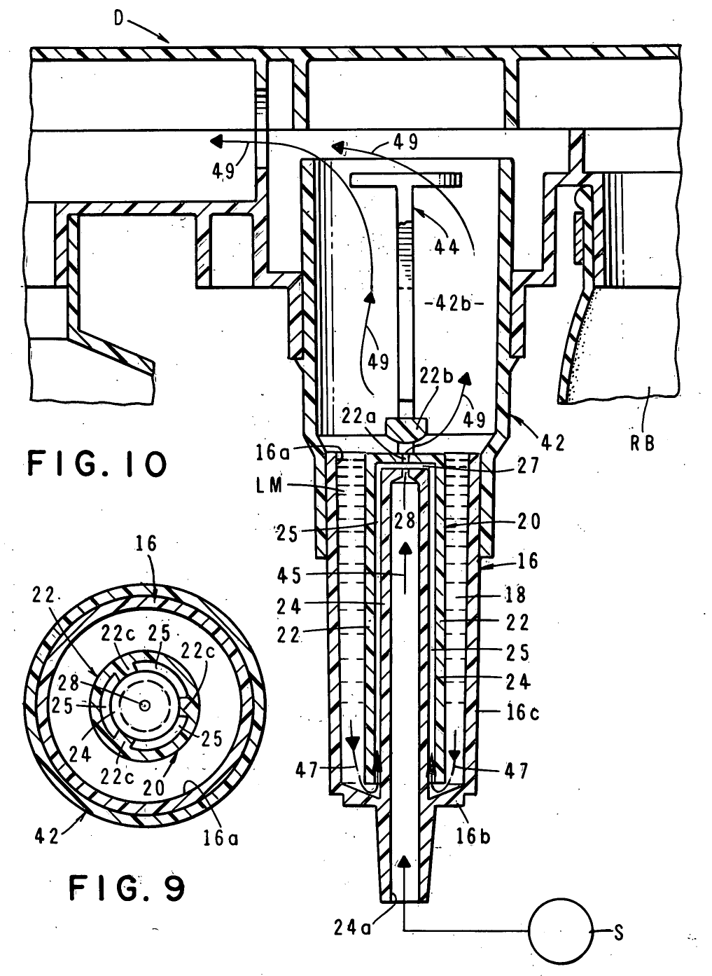

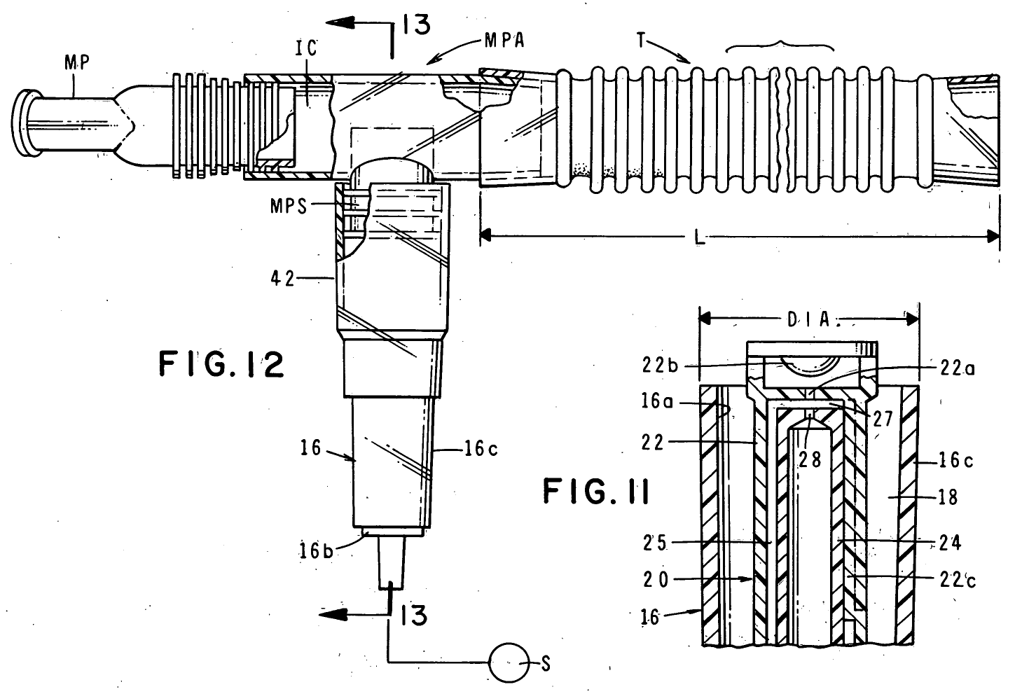

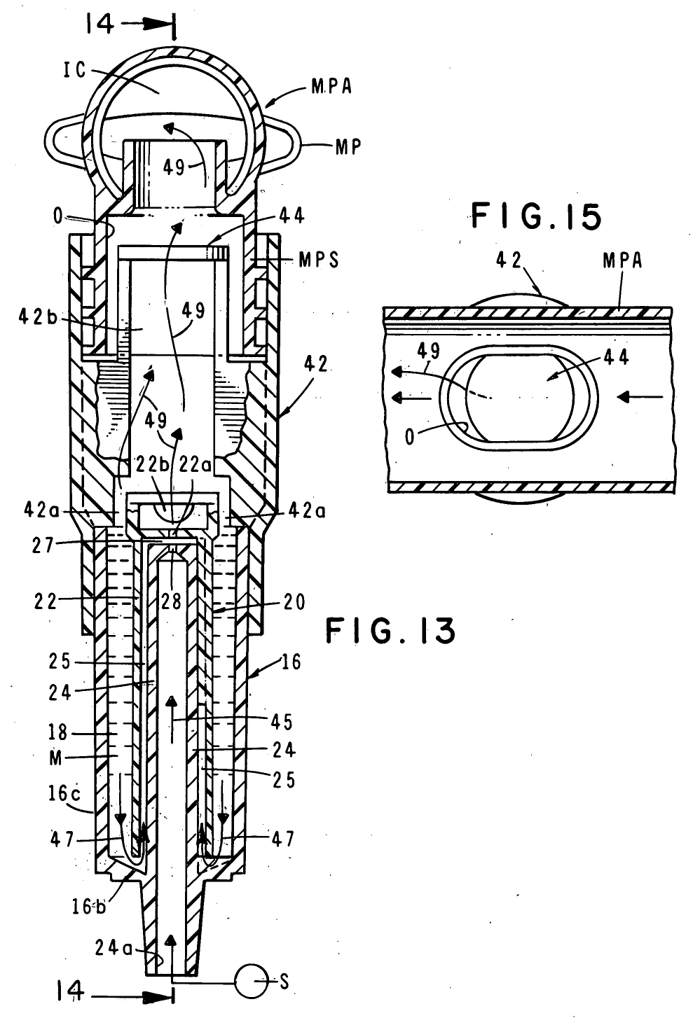

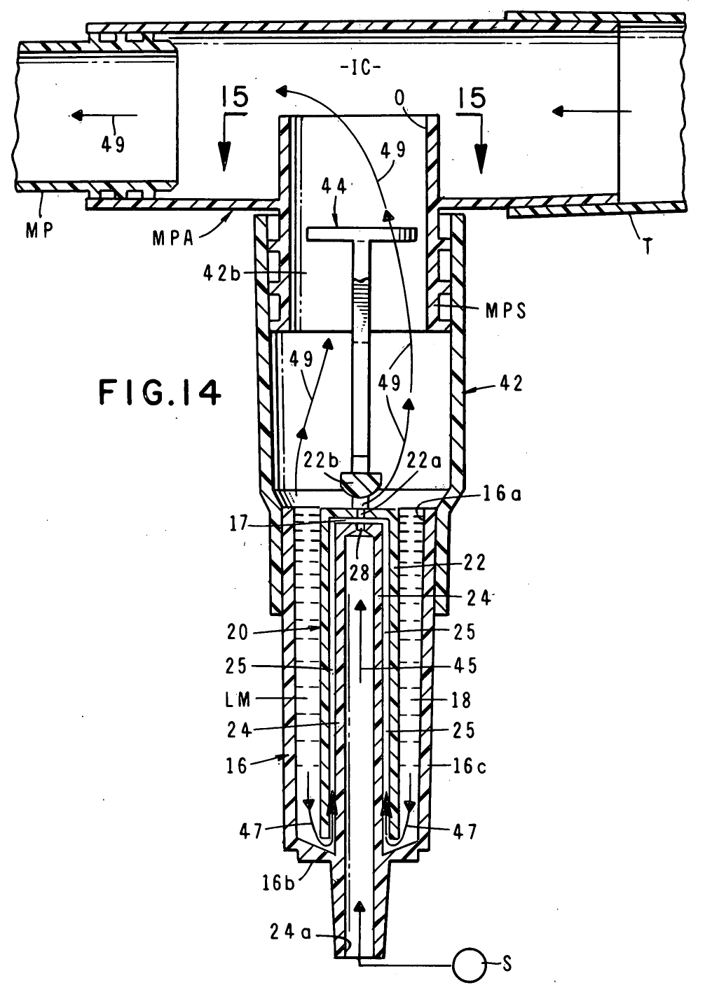

As best seen by referring to FIGS. 2, 3 and 9, nebulizer body 22 is telescopically receivable over flow tube 24 and includes a plurality of circumferentially spaced ribs 22c that cooperate with the outer wall of the flow tube to define a plurality of fluid flow paths 25 (FIG. 9). When the nebulizer body is in position over the flow tube, the components cooperate to define a transverse fluid passageway 27 that is in communication with the plurality of fluid flow passageways 25 and with gas outlet port 28. With this construction, when the reservoir 18 is filled with the aerosolizable liquid medicament “LM” and when the fluid flow tube 24 is interconnected with a source of gas under pressure “S” (FIG. 4), the aerosolizable liquid medicament “LM” will, in a manner presently to be described, be aerosolized to produce a multiplicity of particles of aerosolized medication.

Removably connected to central body 16 is a bottom closure assembly 26 that includes a supporting base 29 and an elongated stem 30 that is connected to supporting base 29 in the manner best seen in FIG. 3 of the drawings. As indicated in the drawings, elongated stem 30 is telescopically, sealably receivable within the fluid flow tube 24 for sealing the gas inlet port 24a thereof. In one form of the present invention, supporting base 29 functions to enable proper positioning of nebulizer 14 for automated robotic filling procedures. In this regard, it should be noted that the overall design of the nebulizer unit of the present invention is such that it is fully compatible with an automated robotic assembly process, with automated robotic post-assembly functional testing and quality assurance inspection, and with automatic robotic packaging processes for packaging and shipping the assembled unit in a fashion that meets the needs of the pharmaceutical companies.

Removably connected to first open end 16a of central body 16 is a top closure assembly 32 that comprises a part of the fill means of the invention for filling reservoir 18 with a suitable liquid medicament. Top closure assembly 32 functions to close the first open end of the central body and also functions to enable the reservoir 18 to be filled with the aerosolizable liquid medicament “LM”. As best seen in FIG. 3 of the drawings, assembly 32 comprises a closure cap 34 that includes a top wall 34a and a tapered skirt portion 34b that is connected to the top wall and depends therefrom. Tapered skirt portion 34b includes a reduced diameter portion 35 that is sealably receivable within open end 16a of central body portion 16 in the manner illustrated in FIG. 2 of the drawings. This is but one form of closure that was designed into the working prototype to demonstrate proof of concept. Many other forms of closure are contemplated and the unit is intentionally made adaptable to different closure methodologies as will be required by different pharmaceutical companies.

Top wall 34a of the closure 34 is provided with an aperture 37 that sealably receives an elastomeric plug 38. As indicated in FIG. 3, aperture 37 can be traversed by the needle “N” of the automated filling apparatus (not shown) that contains the liquid medicament that is to be used to fill the reservoir 18. This is but one form of filling that was designed into the working prototype to demonstrate proof of concept. Many other forms of filling are contemplated based upon the variety of automated filling machinery presently available in the pharmaceutical industry.

After receipt of the requisite number of units by the pharmaceutical company, the units can be filled with a suitable liquid medicament by means of an automated robotic filling process, as previously mentioned, thereby rendering them “pre-filled” in the perspective of the end-user.

Elongated stem 30 effectively seals elongated fluid tube 24 against leakage of liquid medication through 28 after filling and during any subsequent transport of the packaged pre-filled nebulizers before they are used. Immediately prior to use, bottom closure assembly 26 is manually twisted and removed thereby withdrawing the stem 30 from the fluid tube 24 thus readying the device for use. Bottom closure assembly 26 is now discarded.

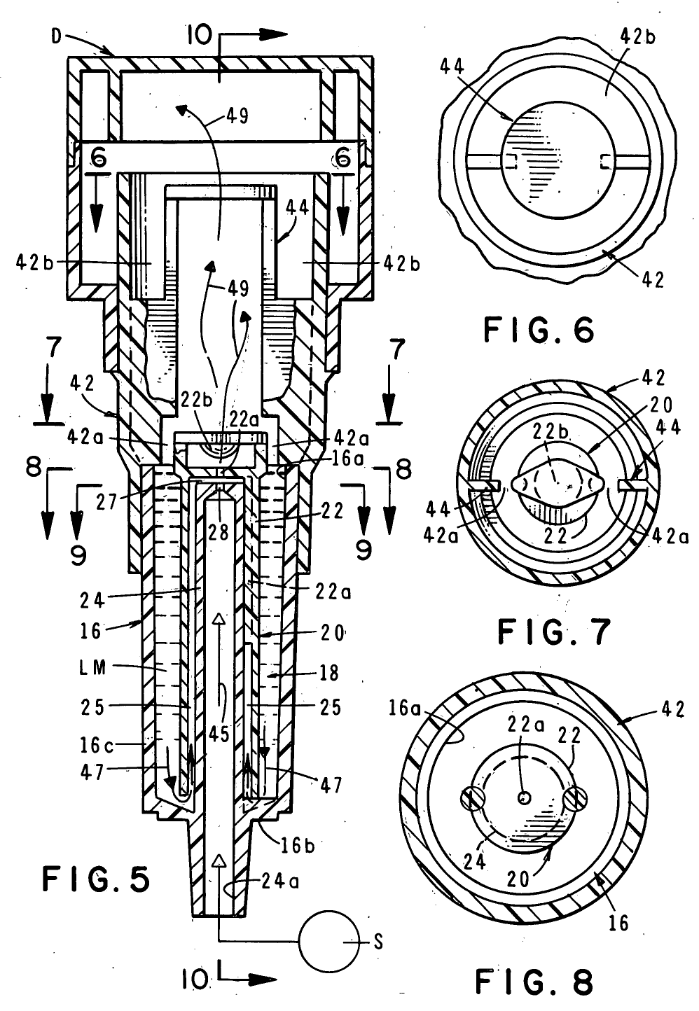

Turning now to FIG. 5 of the drawings, when the nebulizer unit of the present invention is to be used with a dosimetric patient delivery device “D”, such as that described in U.S. Pat. No. 5,727,542 issued to one of the present inventors, the bottom closure assembly 26 is removed and discarded, then the top closure assembly 32 is removed from the central body portion 16 and an injection molded connector adapter 42 is connected to the central body portion in the manner illustrated in FIGS. 5 and 10 of the drawings. Connector adapter 42 includes inlet ports 42a that are in communication with an expansion chamber 42b for expanding the plume of driving gas and decelerating the multiplicity of particles of aerosolized medication emitted from the nebulizer orifice 22a.

As best seen in FIGS. 5, 7 and 10 of the drawings, connector adapter 42 further includes an internal baffle assembly 44 for reducing the size of the multiplicity of particles of aerosolized medication reaching the patient delivery device. In this regard, the volume of expansion chamber 42b must be sufficiently large to enable the aerosol-laden gas plume emitting from the nebulizer orifice 22a to sufficiently re-expand and for the multitude of aerosol particles produced to decelerate in order that the larger particles deliberately encounter the baffling effects of the device and recombine into liquid droplets which are recycled through the nebulizer while the smaller, respirable, particles are effectively emitted from the nebulizer and carried forward to the patient by the gas flow through the nebulizer.

While in the preferred embodiment of the invention, the expansion, or deceleration, chamber 42b is provided as a separate component, that can be conveniently attached to the selected patient delivery interface in series with the nebulizer unit, the deceleration chamber can, for particular end use requirements be incorporated into the basic nebulizer unit.

The dosimetric patient delivery device “D”, such as that described in U.S. Pat. No. 5,727,542, when coupled with the nebulizer 14 in a manner illustrated in FIGS. 4, 5 and 10 of the drawings, provides the ability to semi-quantitize the patient dose and deliver a drug with such efficiency that often the patient inhalation time to receive a required dose of medication can be reduced to a fraction of that now required using prior art inhalation devices. This novel combination delivers superior patient dose consistency and repeatability even over a wide range of patient breathing parameters. It also provides the ability to accurately predict the actual dose delivered to the patient within ±20% of that predicted when using the dose quantification equation presently to be discussed. Because of its pertinence, U.S. Pat. No. 5,727,542 is hereby incorporated by reference as though fully set forth herein.

Operationally the device described and incorporated by reference patent '542 includes a novel, almost resistance-free flapper valve mechanism which directs the output of nebulizer 14 to the patient upon inhalation, and into a reservoir bag “RB” during the period of patient exhalation (FIG. 4). That aerosol which is temporarily retained in the bag becomes additional medication for the patient upon the next inhalation and supplements the delivery of medication provided by real-time operation of the nebulizer rather than being shunted out through the expiratory pathway. Typically in conventional, prior art nebulizer devices, the medication aerosolized during the patient's expiratory phase is lost to the atmosphere and essentially wasted. The immediate obvious benefits of the system of the present invention as illustrated in FIGS. 4, 5 and 10 of the drawings are: (1) drug delivery efficiency is increased dramatically (on average by a factor of 2.4 times); (2) far less medication is wasted by release to the atmosphere; (3) if oxygen is used as the driving gas to power the nebulizer, the fraction of inspired oxygen (FIO₂) provided to the patient will be maintained at a high level.

A less-obvious secondary benefit of the invention resides in the more consistent and reproducible dosing quantities to the patient. If the actuating flow of oxygen or air to the nebulizer unit is in the region of 6 or 7 liters per minute (L/min), and if the patient's minute ventilation (tidal volume multiplied by respiratory rate) is essentially the same as the actuating flow, use of the system of the present invention minimizes very greatly changes in drug delivery due to differing breathing patterns. Inasmuch as these operating parameters closely match typical human breathing patterns, this system will accommodate a range of patients from pediatrics through adults. In this regard, experience has shown that the system, when used with adult or semi-adult patients, will maintain dose repeatability to within +20%.

Further, from the Dose Quantification equation, presently to be discussed, it is obvious that the patient delivered dose of medication is directly proportional to the drug concentration (mg/mL) being aerosolized and the treatment time, all other factors being constant. Therefore, by proper selection of the drug concentration in the pre-filled nebulizer unit, and regulation of the treatment time, the desired doses can be delivered to the patient in as little as one-minute of treatment time.

In using the apparatus of the invention in connection with a dosimetric patient delivery device “D”, the top closure assembly 32 is disconnected from the body portion 16 and the connector adapter 42 is interconnected with the inlet port of the dosimetric patient delivery device “D” in the manner illustrated in FIGS. 5 and 10 of the drawings. This done, the bottom closure assembly 26 is removed from the elongated fluid flow tube 24 thereby exposing the gas inlet port 24a. Next, the fluid flow tube 24 is interconnected with the source of gas under pressure “S” (FIG. 4). The gas is preferably supplied to the nebulizer from the source “S” at a flow rate of about 6 to 7 liters per minute. As illustrated in FIG. 5, the gas flowing through the gas inlet port 24a in the direction of the arrow 45 passes through the very small nebulizer orifice 28 provided in the nebulizer body 24. As the gas courses upwardly through the fluid flow tube 24, it creates a partial vacuum in the circumferentially spaced fluid passageways 25. This vacuum causes the level of the liquid medicaments in the reservoir 18 to flow into passageways 25 in the direction of the arrow 47 and then to flow over the top of the fluid passageways 25. Due to the basic design of the nebulizer and in accordance with the Bernoulli effect, when the stream of gas flowing through the fluid flow tube strikes the liquid drawn from the reservoir it will be predictably converted into a fine mist containing a mixture of particles of aerosolized medication of varying sizes that will be carried upwardly through spray orifice 22a formed in plastic nebulizer body 22. In the present form of the invention, the nebulizer orifice produces a multiplicity of particles comprising larger particles of a size exceeding 5 microns and smaller particles of a size between 0.2 to 5 microns.

After flowing through orifice 22a, the fine particulate-laden mist following impact with the selector element 22b will flow into expansion chamber 42b of the connector adapter 42 and around and about baffle 44 in the direction of the arrows 49 in a manner to decelerate the multiplicity of particles of aerosolized medication emitted from the nebulizer orifice 22a. This deceleration of the particles reduces the size of the particles reaching the outlet port of the device and limits the size of the particles that ultimately reach the patient.

Through use of the combination nebulizer and dosimetric patient delivery device “D” as described in the preceding paragraphs, the following Dose Quantification equation can be effectively used in predicting the delivered patient dose [Inhaled Mass, predicted (IMp)]:

IMp=C×AGR×K×SE×T

where:

IMp: Inhaled Mass, predicted (mg)

C: Drug Concentration (mg/mL)

AGR: Aerosol Generation Rate, i.e., the rate of conversion of liquid to aerosol, (mL/min)

K: AGR Constant; fractional multiplier representing the typical drug content as fraction of AGR

SE: System Efficiency; fractional multiplier representing the System Efficiency (i.e., percentage of output drug/nebulizer output)

T: Time of aerosolization, i.e., Treatment Time, (minutes)

### EXAMPLE

Calculate predicted dose to patient when aerosolizing a medication, such as albuterol, with a concentration [C] of 5 mg/mL for one minute with an AGR of 0.25 and a constant [K] of 0.5 and SE of 0.65.

IMp=5×0.25×0.5×0.65×1=0.32 mg

An Inhaled Mass (delivery) of 0.32 mg of albuterol is typical of the nominal dose of albuterol delivered by most conventional prior art small volume nebulizers.

Turning now to FIGS. 12, 13 and 14, the nebulizer unit of the invention is there shown interconnected with a different form of patient delivery device, here shown as a conventional mouthpiece and tee connector assembly “MPA” that comprises a corrugated aerosol reservoir flex tubing “T” having a length “L” and a conventional mouthpiece “MP”.

As best seen in FIG. 14, assembly “MPA” is provided with a skirt “MPS” having inlet opening “O”, To interconnect the nebulizer unit of the invention with the mouthpiece assembly the connector adapter 42 is telescopically received over the skirt “MPS” in the manner depicted in FIG. 14.

Following removal of the lower closure assembly 26, the fluid flow tube 24 is interconnected with the source of gas under pressure “S” (FIGS. 12, 13 and 14). As illustrated in FIG. 14, the gas flowing through the gas inlet port 24a in the direction of the arrow 45 passes through the very small nebulizer orifice 28 provided in the nebulizer body 24. As before, as the gas courses upwardly through the fluid flow tube 24 it creates a partial vacuum in the circumferentially spaced fluid passageways 25. This vacuum causes the level of the liquid medicaments in the reservoir 18 to flow into passageways 25 and then to flow over the top of the fluid passageways 25. Due to the basic design of the nebulizer, when the stream of gas flowing through the fluid flow tube strikes the liquid drawn from the reservoir it will be predictably converted into a fine mist containing a mixture of particles of aerosolized medication of varying sizes that will be carried upwardly through spray orifice 22a formed in plastic nebulizer body 22.

After flowing through orifice 22a, the fine particulate-laden mist following impact with the selector element 22b will flow into expansion chamber 42b of the connector adapter 42 and around and about baffle 44 in the direction of the arrows 49 in a manner to decelerate the multiplicity of particles of aerosolized medication emitted from the nebulizer orifice 22a. The particles of aerosolized medication will then flow to the internal chamber “IC” of the mouthpiece, along the length of the mouthpiece, outwardly of the mouthpiece outlet and into the mouth of the patient.

By way of summary, the several advantages of the apparatus of the present invention for hospitals and home care agencies include the following:

1. Patient delivery time using the combination nebulizer 14 and dosimetric patient delivery device “D” for the delivery of albuterol and similar inhalable drugs can be reduced to about a one-minute treatment time.
2. Influence of patient breathing pattern on drug delivery can be substantially minimized or negated.
3. Single-use “throw-away” technology embodied in the apparatus of the invention completely eliminates the need for post-treatment nebulizer cleaning and removes all doubt about the effectiveness of this procedure.
4. Within limits, through use of the apparatus of the invention, patient dose is reasonably quantifiable and predictable.
5. Total time for hospital patient treatments can be greatly reduced, through reduction of both pre-treatment set-up time and post-treatment clean-up time, thereby resulting in both labor-savings and cost-savings for the facility.
6. The use of the apparatus of the invention, in combination with the dosimetric patient delivery device “D,” substantially reduces atmospheric contamination to less than approximately 15% of nebulizer loading dose.
7. The very short treatment time of approximately one minute contributes to improved patient compliance, especially with patients receiving multiple inhalation drugs.
8. Particle size control can be readily built into the nebulizer design; that is, different particle baffling designs can be made available for different desired particle sizes as characterized by measurements of mass median aerodynamic diameter (MMAD).
9. Device acquisition cost to the healthcare facility or home care agency is substantially or completely offset by labor savings in the hospital environment and probable reduction in service or maintenance calls for home care patients undergoing self-treatment in the home environment.

Having now described the invention in detail in accordance with the requirements of the patent statues, those skilled in this art will have no difficulty in making changes and modifications in the individual parts or their relative assembly in order to meet specific requirements or conditions. Such changes and modifications may be made without departing from the scope and spirit of the invention, as set forth in the following claims:

We claim:

1. A single dose nebulizer for delivering a multiplicity of particles of aerosolized medication of a selected size to a patient comprising:
(a) a central body having a first open end, a second end and a reservoir containing an aerosolizable liquid medicament;
(b) a nebulizer assembly disposed within said reservoir for converting said aerosolizable liquid medicament into an aerosolized medication, said nebulizer assembly comprising:
(i) a nebulizer body having a first open end, a second end and a nebulizer orifice; and
(ii) a fluid flow tube connected to said second end of said central body, said fluid flow tube having a gas inlet port and a gas outlet port in communication with said nebulizer orifice for aerosolizing said aerosolizable liquid medicament to produce a multiplicity of particles of aerosolized medication; and
(c) a bottom closure assembly removably connected to said central body, said bottom closure assembly including a supporting base and an elongated stem connected to said supporting base and sealably receivable within said fluid flow tube for sealing said gas inlet port of said fluid flow tube; and
(d) a connector adapter which includes an expansion chamber for deceleration of said multiplicity of particles of aerosolized medication emitted from said nebulizer orifice.

2. The nebulizer as defined in claim 1 in which said reservoir of said central body contains between 2 and 4 milliliters of aerosolizable liquid.

3. The nebulizer as defined in claim 1 in which said nebulizer orifice produces a multiplicity of particles comprising larger particles of a size exceeding 5 microns and smaller particles of a size between 0.2 to 5 microns.

4. The nebulizer as defined in claim 1 in which said central body has a diameter of between about 0.5 and about 0.8 inches.

5. The nebulizer as defined in claim 1 in which the overall length of said nebulizer is between about 2.0 and about 3.0 inches.

6. The nebulizer as defined in claim 1 in which said nebulizer is injection molded from a commercial polymer.

7. The nebulizer as defined in claim 1, further including a top closure assembly connected to said first end of said central body.

8. The nebulizer as defined in claim 1, further including a connector adapter connected to said first end of said central body.

9. The nebulizer as defined in claim 8 in which said connector adapter functions to interconnect said central body portion with a patient mouthpiece, tee connector and corrugated aerosol tubing.

10. The nebulizer as defined in claim 8 in which said connector adapter may be further connected to a so-called “valved tee adapter” for connection into the tubing circuit of a mechanical ventilator.

11. The nebulizer as defined in claim 8 in which said connector adapter functions to interconnect said central body portion with a dosimetric patient delivery device.

12. The nebulizer as defined in claim 11 in which said connector adapter further includes an internal baffle for reducing the size of the multiplicity of particles of aerosolized medication reaching said outlet port of said patient delivery device.

13. A single dose, disposable nebulizer for delivering a multiplicity of particles of aerosolized medication of a selected size to a patient comprising:
(a) a central body having a first open end, a second end and a reservoir containing an aerosolizable liquid medicament;
(b) a nebulizer assembly disposed within said reservoir for converting said aerosolizable liquid medicament into an aerosolized medication, said nebulizer assembly comprising:
(i) a nebulizer body having a nebulizer orifice; and
(ii) a fluid flow tube connected to said second end of said central body, said fluid flow tube having a gas inlet port and a gas outlet port in communication with said nebulizer orifice for aerosolizing said aerosolizable liquid medicament to produce a multiplicity of particles of aerosolized medication; and
(c) a bottom closure assembly removably connected to said central body, said bottom closure assembly including a supporting base and an elongated stem connected to said supporting base and sealably receivable within said fluid flow tube for sealing said gas inlet port of said fluid flow tube.

14. The nebulizer as defined in claim 13, further including a connector adapter connected to said first end of said central body for connecting said central body to a patient delivery device.

15. The nebulizer as defined in claim 13, further including a top closure assembly removably connected to said central body, said top closure assembly comprising a top wall and a downwardly depending skirt connected to said top wall, said skirt being sealably receivable over said central body.

16. The nebulizer as defined in claim 11 in which said top wall of said top closure assembly is provided with a filling aperture.

17. The nebulizer as defined in claim 11 in which said reservoir of said central body contains between 2 and 4 milliliters of aerosolizable liquid.

18. The nebulizer as defined in claim 11 in which said central body has a diameter of between about 0.5 and about 0.8 inches.

19. The nebulizer as defined in claim 11 in which the overall length of said nebulizer is between about 2.0 and about 3.0 inches.

20. A single dose miniaturized, disposable nebulizer for delivering a multiplicity of particles of aerosolized medication of a selected size to a patient comprising:
(a) a molded plastic central body having a diameter less than about 0.7 inches, said central body having a first open end, a second end and a reservoir for containing between about 2 and about 4 milliliters of aerosolizable liquid medicament;
(b) a fluid flow tube connected to said second end of said central body, said fluid flow tube having a gas inlet port and a gas outlet port;
(c) a nebulizer assembly disposed within said reservoir of said central body for converting said aerosolizable liquid medicament contained within said reservoir into a multiplicity of particles of aerosolized medication, said nebulizer assembly comprising a moldable plastic nebulizer body having a nebulizer orifice; and
(d) a bottom closure assembly removably connected to said central body, said bottom closure assembly including a supporting base and an elongated stem connected to said supporting base, said elongated stem being sealably receivable within said fluid flow tube for sealing said gas inlet port of said fluid flow tube; and
(e) a connector adapter connected to said central body for interconnecting said central body with a patient delivery device having an outlet port in communication with the patient, said connector adapter including an expansion chamber for deceleration of said multiplicity of particles aerosolized medication emitted from said nebulizer orifice and an internal baffle disposed within said expansion chamber for reducing the size of the multiplicity of particles of aerosolized medication reaching said outlet port of said patient delivery device.
